(19) United States
(12) Patent Application Publication
(10) Pub. No.: US 2010/0234877 A1
(43) Pub. Date: Sep. 16, 2010

(54) ELECTROMECHANICAL TOURNIQUET FOR BATTLEFIELD APPLICATION

(76) Inventors: David Pienkowski, Lexington, KY (US); Lee Gentry Barnett, Lexington, KY (US)

Correspondence Address: KING & SCHICKLI, PLLC, 247 NORTH BROADWAY, LEXINGTON, KY, 40507, US

(21) Appl. No.: 12/424,200

(22) Filed: Apr. 15, 2009

Related U.S. Application Data
(60) Provisional application No. 61/045,440, filed on Apr. 16, 2008.

(51) Int. Cl. A61B 17/132

(52) U.S. Cl. 606/203

(57) ABSTRACT

An electromechanical tourniquet includes a strap, a buckle assembly connected to the strap and utilized to secure and tighten the strap around a limb of a person and a pressure pad carried on the strap. The pressure pad provides localized application of pressure along a line extending into the limb of a person to whom the tourniquet is applied. The tourniquet further includes a force sensor to measure occlusive pressure applied to the limb along that line as well as a user interface that displays duration of use.

This application claims the benefit of U.S. Provisional patent application Ser. No. 61/045,440 filed on 16 Apr. 2008.

### TECHNICAL FIELD

The present invention relates generally to the emergency medical equipment field and, more particularly, to a new and improved electromechanical tourniquet and to a method of confirming that a tourniquet is providing desired occlusive pressure to a limb of a person to which it is applied.

### BACKGROUND OF THE INVENTION

The term extremity injury is used to describe any biological damage to the body's arms and legs. Extremity injuries can result from a wide variety of causes and can range in severity from a negligible skin wound to a life-threatening laceration or crush. Severity of an extremity injury depends on the location and path of the injury. Critical results are commonly associated with compromised vascular components of the particular extremity.

Blood is supplied to the arm through the brachial artery which branches into the radial artery and ulnar artery near the elbow. In the leg, the iliac artery traveling distal from the pelvic region becomes the common femoral artery after maneuvering under the inguinal ligament. Near the head of the femur, the common femoral artery then branches into the femoral profunda (deep femoral artery) and the superficial femoral artery.

The deep femoral artery travels medially down the thigh in close proximity to the femur. Along its path, the deep femoral artery branches in perforating arteries, which supply blood to the femur and musculature of the thigh.

The superficial femoral artery travels the length of the thigh near the border. After passing through the adductor hiatus of the knee, the superficial femoral artery becomes the popliteal artery which supplies blood to the lower leg.

Vascular injuries to the extremities become critical when life threatening blood loss is possible. The threshold for life threatening blood loss occurs when one-half of one's total blood volume is lost. Based upon average human blood volume of 6L, this critical blood loss is 3L.

TABLE 1
Time to 3 L Blood Loss

| | Activity | Flow Rate | Time to 3 L Loss |
|---|---|---|---|
| Vascular Component | Level | (L/min) | (min) |
| Brachial Artery (Arm) | — | 0.10 | 30.0 |
| Common Femoral Artery | Rest | 0.284 | 10.6 |
| | Mild | 1.00 | 3.0 |
| | Moderate | 1.50 | 2.0 |
| Superficial Femoral Artery | — | 0.152 | 19.7 |
| Deep Femoral Artery | — | 0.132 | 22.7 |
| Popliteal Artery | — | 0.072 | 41.7 |

Table 1 shows estimated time to 3L blood loss for major arteries in the extremities. Activity level is shown to have a substantial impact in time to 3L loss. A common femoral artery injury to a victim at rest can result in 3L loss in approximately 10 minutes whereas the same injury to a victim undergoing moderate activity occurring in approximately 2 minutes. Table 1 also concludes that vascular injuries to the major arteries of the upper leg (common femoral artery, deep femoral artery, and superficial femoral artery) result in 3L loss at least 10 minutes quicker than vascular injuries to the lower leg and arm. This is due to greater blood flow rate in these arteries.

Even though civilian extremity injuries are common, extremity injuries occurring in the battlefield are more frequent. The increased probability of an extremity injury on the battlefield is due to the minimal protection of a soldier's extremities. Today's typical soldier armor includes chest and head protection, but does not include shielding of the upper and lower extremities due to a soldier's demand for mobility. This defensive exclusion has allowed extremity injuries to become the most prevalent injury to American soldiers.

When a major artery is severed, either by injury or surgical intervention, controlling blood loss becomes vital. In severe cases, where potential blood loss is considered life-threatening, a tourniquet may be applied proximal to the vascular disruption to manage blood loss. Because of its rapid blood flow occlusion capabilities, the tourniquet is commonly considered a life-saving device in emergency situations. It is also used to create a controlled “bloodless” operating area in surgical procedures.

A tourniquet can be described as a restrictive device that occludes blood flow. Standard tourniquets apply circumferential pressure to the skin which is transferred to the underlying tissues and associated vascular components. As the pressure applied by the tourniquet increases, the pressure applied to the arteries and veins increases. The diameter of the vascular components decreases due to the applied pressure. The decreased diameter results in decreased flow.

Emergency tourniquets, as the name suggests, are for emergency applications in which the victim of severe limb injury cannot reach a medical facility immediately. Tourniquets are not necessarily considered part of a standard first aid kit, but in unforeseen critical incidents, the application of an emergency tourniquet can be the deciding factor between life and death. Emergency tourniquets must be adjustable in size to fit a range of possible users, collapsible to take up little space, and easy to use so that a user can apply it to him/herself in a critical situation. These tourniquets can be essential in many emergency situations: a tourniquet may be applied at the scene of a car accident if the victim has a serious injury and is trapped in a car; a tourniquet may be applied at the scene of a rural accident such that blood loss is reduced until medical help arrives; a tourniquet may be applied at the scene of a snake bite incident to prevent poisonous toxins from traveling to other parts of the body; and a tourniquet may be self-applied during a hiking or mountain climbing accident to prevent blood loss until medical help arrives. Although emergency tourniquets are vital in accident survival, the most common use of emergency tourniquets is in the military.

Military tourniquets are simply emergency tourniquets specialized for use on the battlefield. The goal of military tourniquets is to extend the survival time until a soldier can reach additional medical aid.

The present invention relates to a new and improved emergency tourniquet that is particularly suited for use by the military. The electromechanical tourniquet of the present invention was developed with the capability of monitoring application time, producing effective occlusive pressure and monitoring the occlusion pressure. Knowledge of tourniquet application time serves as a determining factor for the method of treatment once an individual reaches a medical facility. Specifically it allows medics to estimate the amount of blood loss and the extent of the extremity injury. Based upon tourniquet application time and other hard signs, primarily hematoma, hemorrhage, or acute ischemia, the patient will be diagnosed and the severity of injury will be scored using the injured extremity index. Monitoring application time is also important with regard to the side effects of tourniquet application and may also play a part in determining the extent of an extremity injury.

Tourniquet application time is also important to account for readjustment periods. A tourniquet must remain extremely taught to prevent severe blood loss. However, extreme pain is commonly associated with blood occlusion caused by a tourniquet. The pain, caused by hypoxia becomes so extensive in certain cases that some victims loosen the tourniquet to allow some blood flow distal to the tourniquet application site which therefore alleviates some of the pain. The individual later retightens the tourniquet. This process may occur multiple times over the period of transportation to a medical facility. However, when the individual reaches medical help this information is rarely transferred to the medics even though the non-uniform occlusion may alter the treatment plan.

The pressure applied by an emergency tourniquet is controlled by the user. Pain associated with tourniquet application or accompanying upper extremity injury, may prevent an individual from tightening the tourniquet to the proper stress. Also, the tourniquet user may not be aware of the strength needed to occlude severe blood loss.

For each anatomical location and blood flow circumstance, there exists a “correct” pressure associated with proper tourniquet use. If the tourniquet is not tightened appropriately, blood flow will not be sufficiently occluded to prevent severe blood loss. However, if the tourniquet is tightened to far past this value, the tourniquet will place unnecessary pressure on surrounding tissues and nerves. Advantageously, the electromechanical tourniquet of the present invention incorporates the capability of signaling the user regarding the appropriate pressure and monitoring the occlusion pressure.

### SUMMARY OF THE INVENTION

In accordance with the purposes of the present invention as described herein, an electromechanical tourniquet is provided. The electromechanical tourniquet includes a strap, a buckle assembly connected to the strap and utilized to tighten and secure the strap around a limb of a person and a pressure pad carried on the strap. The pressure pad provides localized application of pressure along a line extending into the limb of a person to whom the tourniquet is applied. In addition, the electromechanical tourniquet includes a force sensor for measuring occlusive pressure applied to the limb along that line and a user interface.

In accordance with additional detailed aspects of the present invention, the tourniquet includes a microcontroller connected to the force sensor as well as a battery and a voltage regulator. In addition the tourniquet includes a system enclosure carried on the strap. The system enclosure holds the microcontroller, the battery and the voltage regulator.

Further describing the invention the user interface includes a power button and a display connected to the microcontroller. Further, the user interface includes an extremity switch connected to the controller. The extremity switch includes an upper leg setting, a lower leg setting and an arm setting.

In addition an activation switch and a timer are connected to the microcontroller. A proper occlusive pressure indicator is connected to the microcontroller.

The pressure pad includes an outer face having a width W₁ and an inner face having a width W₂ where W₁>W₂. The width W₁ to width W₂ has a ratio of between about 8:1 and about 3:2. In one possible embodiment the outer face has a width W₁ of between about 2″ and about 6″ and the inner face has a width W₂ of between about 0.5″ and about 2″. Further, the outer face may be convex.

In accordance with another aspect of the present invention a method is provided of determining if the tourniquet is providing desired occlusive pressure to the limb of a person. The method comprises the steps of providing the tourniquet with a mode selection switch including an upper leg setting, a lower leg setting and an arm setting and measuring occlusive pressure provided by the tourniquet to a limb upon which the tourniquet is applied. Still further, the method includes the steps of comparing the measured occlusive pressure to a baseline occlusive pressure determined by the mode selection switch setting and indicating when the measured occlusive pressure exceeds the baseline occlusive pressure. Still further the method includes the step of localizing application of the occlusive pressure along a line extending into the limb upon which the tourniquet is applied by providing a tourniquet with a pressure pad.

In the following description there is shown and described a preferred embodiment of the invention, simply by way of illustration of one of the modes best suited to carry out the invention. As it will be realized, the invention is capable of other different embodiments and its several details are capable of modification in various, obvious aspects all without departing from the invention. Accordingly, the drawings and descriptions will be regarded as illustrative in nature and not as restrictive.

### BRIEF DESCRIPTION OF THE DRAWINGS

The accompanying drawings incorporated herein and forming a part of the specification, illustrate several aspects of the present invention and together with the description serve to explain certain principles of the invention. In the drawings:

FIG. 1 is a perspective view of the electromechanical tourniquet of the present invention;

FIG. 2 is a detailed perspective view of the pressure pad that is attached on the inside of the strap of the tourniquet illustrated in FIG. 1;

FIG. 3 is a schematic diagram illustrating the interconnection of the electromechanical components of the tourniquet of the present invention; and

FIG. 4 is a schematical diagram illustrating the operation of the microcontroller.

Reference will now be made in detail to the present preferred embodiment of the invention, examples of which are illustrated in the accompanying drawings.

### DETAILED DESCRIPTION OF THE PREFERRED EMBODIMENTS OF THE INVENTION

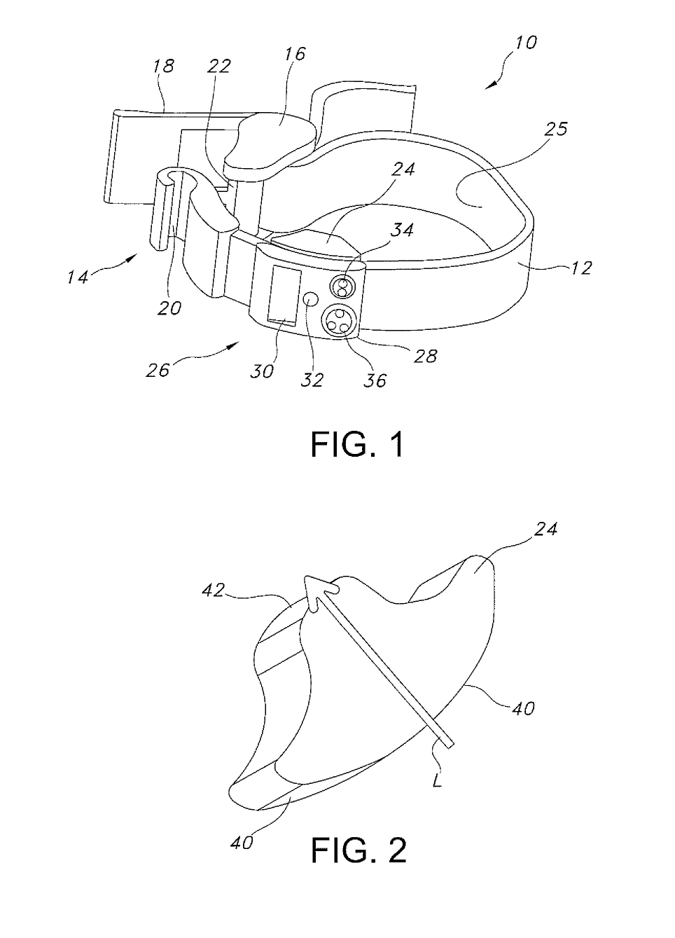

Reference is now made to FIG. 1 illustrating the electromechanical tourniquet 10 of the present invention. The tourniquet 10 includes an elongated strap 12 formed from nylon, such as Cordura brand fabric, or other appropriate material that is preferably strong, lightweight and waterproof as well as easy to clean. A bucket assembly 14 is connected to the strap 12. The bucket assembly 14 comprises a ratchet buckle 16 with an operating lever 18 at one end of the strap 12 and a quick release hook 20 at the other end of the strap 12. In use, the quick release hook 20 is connected to the latching bar 22 and the lever 18 is manipulated to operate the ratchet mechanism of the ratchet buckle 16, thereby shortening the strap 12 and tightening the tourniquet 10 down on the limb of an individual.

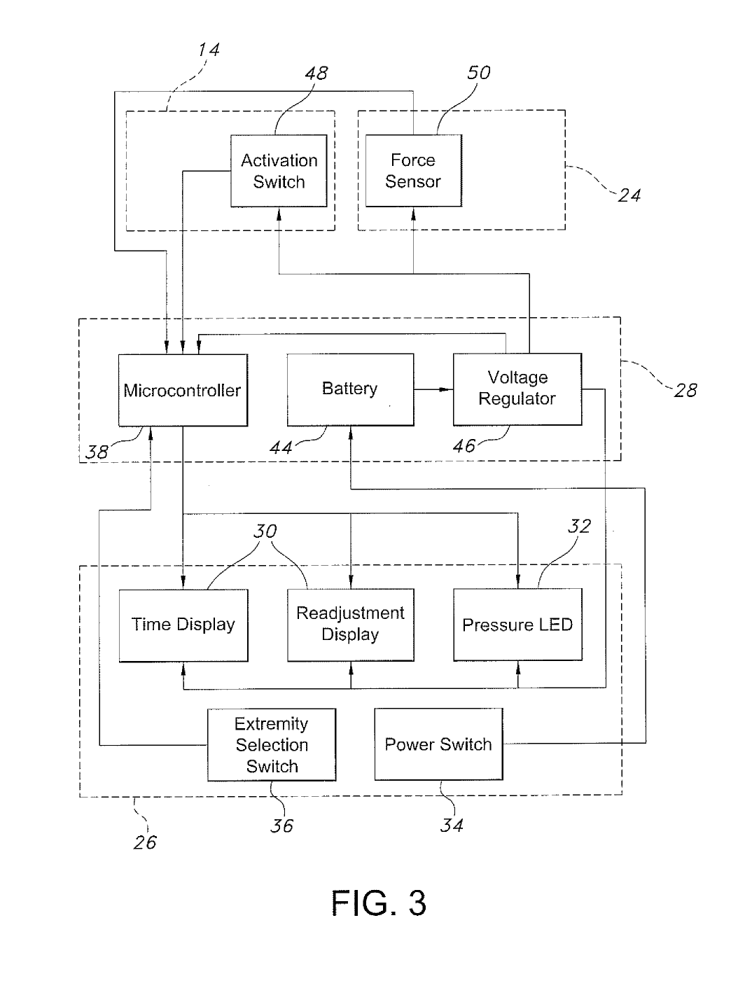

A pressure pad 24 is connected to the limb engaging face 25 of the strap 12. As best illustrated in FIGS. 1 and 2, the pressure pad 24 includes an outer face 40 that is provided in engagement with the interior face 25 of the strap 12 and an opposite inner face 42. The outer face 40 has a width W₁ of between about 2″ and about 6″ while the inner face 42 has a width W₂ of between about 0.5″ and about 2″. Thus, the width W₁ of the outer face is greater than the width W₂ of the inner face. In one particularly useful embodiment the width W₁ compared to the width W₂ has a ratio of between about 8:1 and about 3:2. As illustrated, it should also be appreciated that the outer face 40 is convex in shape.

The pressure pad 24 may be formed from Neoprene, EPDM, polyurethane, silicone rubber or other appropriate material. In use, the pressure pad 24 functions to provide localized application of pressure along a line extending into the limb of the person to whom the tourniquet 10 is applied. The line L is illustrated in FIGS. 2 and 5. As should be appreciated, that line L is provided in alignment with the artery A through which the user desires to occlude blood flow. As illustrated in FIG. 5, the line L extends through the femoral vein and artery F₁ as well as the deep femoral vein and artery F₂ located adjacent the fervor F₃ of a thigh T.

As further illustrated in FIG. 1, the tourniquet 10 includes a user interface generally designated by reference numeral 26. The user interface 26 comprises a waterproof and preferably air tight enclosure 28. The enclosure 28 holds a display such as an LED display 30, a proper occlusion pressure indicator or pressure LED 32, a power button 34 and an extremity selector switch 36. The operation and function of the display 30, pressure LED 32, power button 34 and extremity selector switch 36 will be described in greater detail below. In addition, the system enclosure 28 holds a microcontroller 38 that controls the operation of the tourniquet 10, a battery 44 that powers all electrical components of the tourniquet and a voltage regulator 46 that ensures the proper voltage is provided to each of the electrical components during operation.

The microcontroller 38 may, for example, be a PIC1bF913 manufactured by Microchip. It should be appreciated, however, that any other microcontroller 38 may be used so long as it is appropriate for its intended purpose.

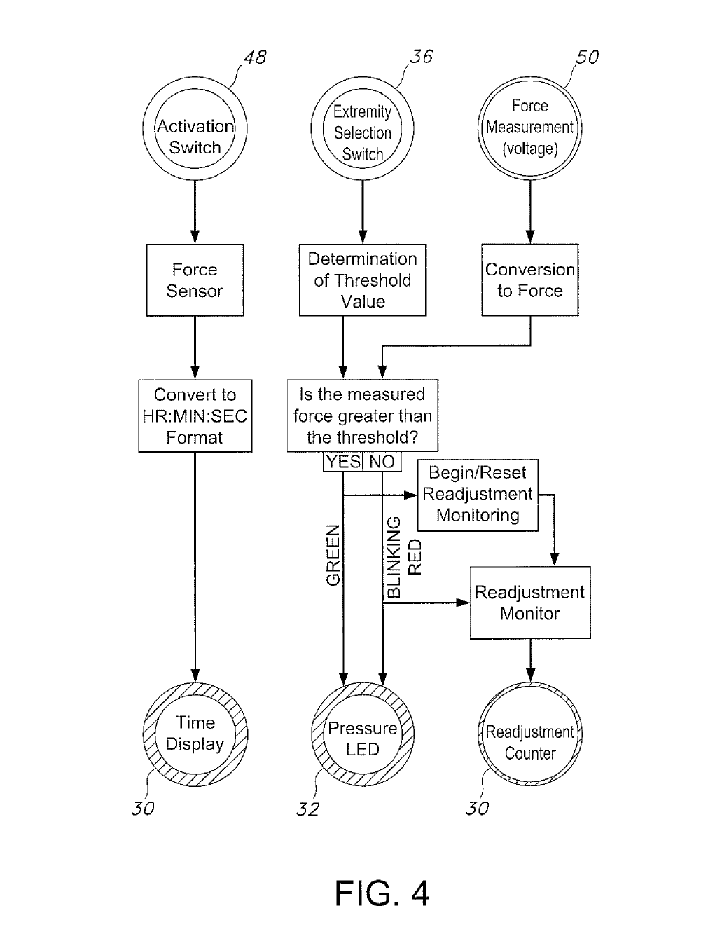

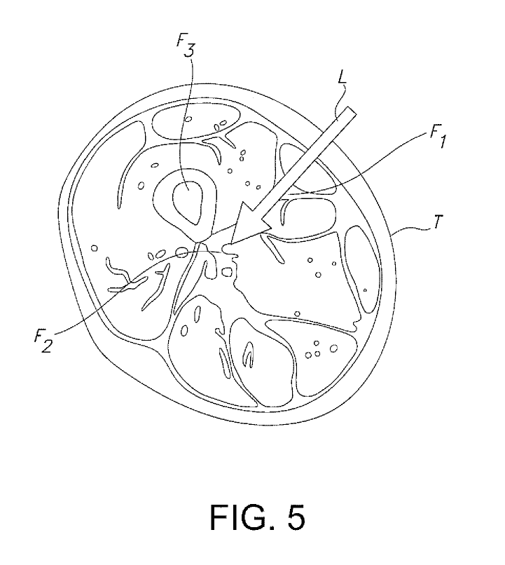

The operation of the tourniquet 10 of the present invention will now be discussed in detail with reference to FIGS. 3 and 4.

First the user identifies the limb, arm or leg, that is injured. The tourniquet 10 is then placed in proper position on the limb above the injury. Specifically, the strap 12 is positioned over the limb between the injury and the torso of the injured individual. The power button 34 is activated and the extremity selector switch 36 is then set.

More specifically, the extremity selector switch 36 is a mode selection switch incorporating three different settings: an upper leg setting, a lower leg setting and an arm setting. One must provide a different occlusive pressure to the upper leg, lower leg and arm in order to properly occlude blood flow. If the tourniquet 10 is applied to an arm, the arm setting is selected. If the tourniquet 10 is applied to a leg above the knee, the upper leg setting is selected. If the tourniquet 10 is applied to the lower leg below the knee, the lower leg setting is selected. When set, an appropriate threshold value signal 60 is sent by the extremity selector switch 36 to the microcontroller 38 and a base-line occlusive pressure is established (see FIG. 4). The significance of this base line occlusive pressure determination will be apparent as the description of the invention proceeds. Next, the hook 20 is secured to the latching bar 22 so that the strap 12 is looped around the limb. Latching bar 22 is connected to the time activation switch 48 and, accordingly, interconnection of the hook 20 with the latching bar 22 initiates timer operation and application time is thereafter indicated on the display 30.

The operating lever 18 of the ratchet buckle 16 is manipulated in a manner known in the art to gradually shorten the strap 12 and thereby tighten the tourniquet 10 around the selected limb. As this is done, the user slides the strap 12 around to position the pressure pad 22 over the extremity or limb's major artery. During this tightening operation the force sensor 50 in the pressure pad 24 continuously measures the occlusive pressure applied to the limb along the line L. The force sensor 50 may comprise a pieozo-resistive sensing device in which resistance is inversely proportional to applied force. The sensor 50 sends an analog voltage signal in range of 0-5 volts to the micro-controller 38 depending upon the force being applied. The micro-controller 38 converts the analog signal into a digital signal. The micro-controller 38 then converts the measured signal from the force sensor 50 into the measured applied force. The measured applied force is then compared to the baseline or threshold value for the specific extremity setting of the extremity selecting switch 36. If the calibrated applied force is less than the threshold or baseline value, the micro-controller 38 activates the pressure LED 32 in a manner to cause it to blink red. If the force is greater than or equal to the desired baseline or threshold value, the micro-controller 38 activates the pressure LED 32 to illuminate green.

As noted above, the force sensor 50 continuously measures the occlusive pressure applied to the limb E. As a result, the micro-controller 38 will cause the pressure LED 32 to illuminate green immediately upon the applied pressure equaling or exceeding the threshold or baseline pressure determined by the setting of the extremity selector switch 36. Thus, when the user sees the pressure LED 32 first illuminate green, the user knows that he has reached the desired occlusive pressure to staunch blood flow through the injured limb. Further, by tightening the strap 12 no further with the ratchet buckle 16, the use of excessive pressure and the potential medical complications that could result are all avoided.

Once activated, the applied occlusive pressure is continuously monitored by the force sensor 50 and the micro-controller 38 continuously compares the applied occlusive force to the baseline or threshold force needed to occlude blood flow as determined by the setting of the extremity selector switch 36. If the applied pressure falls below the threshold pressure at any time, the micro-controller 38 causes the pressure LED 32 to blink red. If desired, an additional audio signal may be provided to further alert the user to the situation. Once notified of the situation, the user uses the operating lever 18 of the ratchet buckle 16 to further tighten the tourniquet 10 until the applied pressure again is equal to or above the threshold pressure and the pressure LED 32 changes from blinking red to green through operation of the micro-controller 38. Whenever readjustment is required to bring the applied occlusive pressure back to or above the threshold value, the micro-controller 38 provides a signal to the display 30 to note readjustment and a readjustment counter and cooperating timer in the display are activated. In this way the number of readjustments and the time since each readjustment may be displayed. This is true whether the readjustment was intentionally performed to allow some blood flow back through the limb temporarily or unintentionally resulted from a loss of applied pressure for other reasons.

Medical personal can more readily assess the condition of a patient based upon all the information provided on the display 30. More specifically, upon reaching the medical personnel, those personnel can review the display 30 to determine the total amount of time the tourniquet 10 has been applied to the limb, as well as if and when any readjustments to tourniquet pressure have been made. Since the force sensor 50, micro-controller 38 and pressure LED 32 operate together to allow the user to maintain the proper or desired occlusive pressure to staunch the blood flow through the artery of the damaged limb, the injured individual is received by the medical personnel in the best possible condition. As a result, survival rates are increased and potential complications from improper tourniquet use are reduced or eliminated.

More specifically, the method of confirming that the tourniquet 10 is providing desired occlusive pressure to staunch blood flow through an artery A of an injured limb comprises (a) providing the tourniquet 10 with a mode selection switch/extremity selector switch 36 including an upper leg setting, a lower leg setting and an arm setting, (b) measuring the occlusive pressure provided by the tourniquet 10 to the limb upon which the tourniquet is applied, (c) comparing the measured occlusive pressure to a baseline or threshold occlusive pressure determined by the mode selection switch setting and (d) indicating when the measured occlusive pressure exceeds the baseline occlusive pressure. The method further includes localizing application of the occlusive pressure along a line extending into the limb upon which the tourniquet 10 is applied by providing the tourniquet with a pressure pad 24.

A method of identifying that the tourniquet 10 has been released and retightened to the limb of a person is also provided. This method includes (a) the monitoring of the activation switch 4S once the tourniquet 10 has been applied, (b) the recording of the number of times the activation switch is deactivated by opening the buckle assembly 14 and (c) the indicating of this count to the user by means of the display 30.

The foregoing description of the preferred embodiments of the present invention have been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The drawings and preferred embodiments do not and are not intended to limit the ordinary meaning of the claims in their fair and broad interpretation in any way.

What is claimed:

1. An electromechanical tourniquet, comprising:
a strap;
a buckle assembly connected to said strap and utilized to secure and tighten said strap around a limb of a person;
a pressure pad carried on said strap, said pressure pad providing localized application of pressure along a line extending into the limb of the person to whom said tourniquet is applied;
a force sensor measuring occlusive pressure applied to said limb along said line; and
a user interface.

2. The tourniquet of claim 1, further including a microcontroller connected to said force sensor.

3. The tourniquet of claim 2, further including a battery and a voltage regulator.

4. The tourniquet of claim 3, further including a system enclosure carried by said strap, said system enclosure holding said microcontroller, said battery and said voltage regulator.

5. The tourniquet of claim 4, wherein said user interface includes a power button and a display connected to said microcontroller.

6. The tourniquet of claim 5, wherein said user interface includes an extremity switch connected to said controller.

7. The tourniquet of claim 6, wherein said extremity switch includes an upper leg setting, a lower leg setting and an arm setting.

8. The tourniquet of claim 7, further including an activation switch and a timer connected to said microcontroller.

9. The tourniquet of claim 8, further including a proper occlusive pressure indicator connected to said microcontroller.

10. The tourniquet of claim 1, wherein said user interface includes a power button and a display connected to said microcontroller.

11. The tourniquet of claim 10, wherein said user interface includes an extremity switch connected to said controller.

12. The tourniquet of claim 11, wherein said extremity switch includes and upper leg setting, a lower leg setting and an arm setting.

13. The tourniquet of claim 12, further including an activation switch and a timer connected to said microcontroller.

14. The tourniquet of claim 1, wherein said pressure pad includes an outer face having a width W₁ and an inner face having a width W₂ where W₁>W₂.

15. The tourniquet of claim 14, wherein width W₁ to width W₂ has a ratio of between about 8:1 and about 3:2.

16. The tourniquet of claim 14, wherein said outer face has a width W₁ of between about 2″ and about 6″ and said inner face has a width W₂ of between about 0.5″ and about 2″.

17. The tourniquet of claim 16, wherein said outer face is convex.

18. The tourniquet of claim 1, wherein said buckle assembly includes a ratchet buckle and a quick release hook.

19. A method of confirming that a tourniquet is providing desired occlusive pressure to a limb of a person, comprising:
providing said tourniquet with a mode selection switch including an upper leg setting, a lower leg setting and an arm setting;
measuring occlusive pressure provided by said tourniquet to a limb upon which said tourniquet is applied;
comparing said measured occlusive pressure to a baseline occlusive pressure determined by said mode selection switch setting; and
indicating when said measured occlusive pressure exceeds said baseline occlusive pressure.

20. The method of claim 19, including localizing application of said occlusive pressure along a line extending into the limb upon which said tourniquet is applied by providing said tourniquet with a pressure pad.

21. A method of identifying that a tourniquet, having an activation switch, has been released and re-tightened to the limb of a person, comprising:
monitoring said activation switch once the tourniquet has been applied;
recording a number of times said activation switch is deactivated; and
indicating this count to the user.
